(19) United States
(12) Patent Application Publication
(10) Pub. No.: US 2012/0017904 A1
(43) Pub. Date: Jan. 26, 2012

(54) Breathing treatment system and method

(76) Inventors: David R. Ratto, Arcadia, CA (US); Richard L. Murray, Upland, CA (US)

(21) Appl. No.: 12/804,612

(22) Filed: Jul. 26, 2010

(51) Int. Cl. A61M 16/16; A61M 16/00

(52) U.S. Cl. 128/203.26; 128/207.18

(57) ABSTRACT

Humidified respiratory gas is supplied to a patient through an open cannula placed within the openings of the patient's nostrils. The open cannula allows respiratory gas to escape the patient's nostrils and thereby reduce back pressure experienced by a patient during the expiratory periods of the patient's breathing cycle. An open cannula configuration also eliminates uncomfortable masks that may otherwise prevent an obstructive breathing disordered patient from effectively using respiratory therapy.

### FIELD

Disclosed subject matter is related to assisted breathing systems and methods.

### BACKGROUND

Breathing disorders, such as obstructive sleep hypopnea and obstructive sleep apnea exact a tremendous health toll on the increasingly aged and weighty American population. Obstructive sleep apnea will also be referred to herein, simply, as apnea. An apnea may be defined, for example, as a complete cessation of airflow for more than 10 seconds; a hypopnea may be defined as a greater than 30% reduction of airflow for the same period of time. Obstructive sleep apnea is a condition in which an afflicted person stops breathing during sleep because their soft palate collapses and blocks their upper airway. It is estimated that between fifteen-million and thirty-million Americans suffer from obstructive sleep apnea. Aging and obesity can contribute to the condition and, as the American population continues to age and become more obese, more and more Americans are expected to suffer from the condition. Although not so prevalent elsewhere, the condition is expected to gain momentum in other countries as those populations age and gain weight. When a person stops breathing, the level of carbon dioxide in their bloodstream increases, and the increased carbon dioxide levels stress the person's cardiovascular system. People with sleep apnea are more likely to suffer from hypertension, diabetes, and heart disease than the general population. Untreated, apnea can lead to intermittent hypoxemia, sleep fragmentation, metabolic dysfunction, and increased cardiovascular morbidity and mortality. Additionally, because their sleep may be interrupted throughout the night, and they may, therefore, be less alert during the day, people suffering from apnea are likely to suffer from hypersomnolence and are more prone to accidents than people who are able to obtain restful sleep.

Recognizing the significant health risks associated with sleep apnea, the medical community has developed a variety of treatments for the affliction. Those treatments range from minimally to extremely invasive and from expensive to very expensive. A dental appliance that forces a patient's lower jaw forward, thereby opening the upper airway, may cost thousands of dollars and may be somewhat effective when used properly. Even if used properly, such an appliance may cause a patient to develop temporomandibular jaw syndrome (TMJ), a painful condition that may require a patient to stop using the appliance and to seek additional treatment for the TMJ. Additionally, patients who have dental appliances often find them uncomfortable and, as a result, don't use them. A patient's lower jaw may be surgically advanced, at the cost of tens of thousands of dollars and a lengthy recovery, in order to open the upper airway. Naturally, any surgical procedure carries with it the attendant risks of infection, negative reaction to anesthesia or other medications, or other complications.

One approach to treating apnea that has met with some success is the application of continuous positive airway pressure (CPAP), which opens a patient's airway by forcing a respiratory gas through the patient's nasal airway passages. The gas is applied through a mask that may cover the patient's mouth and nostrils, or nostrils alone. Such a CPAP system requires a tight seal between the mask and the patient's face. Without a tight seal, the gas pressure within a patient's airway may drop enough to allow the airway to collapse, thereby undermining the effectiveness of the CPAP system. Additionally, escaping gases may tend to dry out a patient's flesh in a region around the gap between flesh and mask, thereby causing a painful cracking of the surrounding skin. A CPAP mask may require bulky straps and bands to hold it in place, under pressure, against a patient's skin, adding to a patient's discomfort. For these and other reasons, many patients, who have been prescribed the use of a CPAP machine, are non-compliant. Some estimate that more than 80% of patients prescribed the use of a CPAP machine do not use the machine; they would rather suffer the deprivations of apnea than deal with the discomfort and inconvenience of a CPAP system.

A relatively low cost, effective, and comfortable system and method for the treatment of obstructive sleep apnea would therefore be highly desirable.

### SUMMARY

In an apparatus and method in accordance with the principles of claimed subject matter, warm, humidified, respiratory gas may be supplied to a patient through an open delivery system for treatment of respiratory conditions, including obstructive sleep apnea, hypopnea, congestive heart failure, or respiratory failure (acute or chronic), for example. Rather than forcing a gas into a patient's respiratory system by sealing a patient's breathing orifices (e.g., nostrils, and, in some cases, mouth) and forcing a gas under pressure into the patient's nostrils, as a conventional closed system (e.g., CPAP system) would, a system in accordance with the principles of claimed subject matter may supply a respiratory gas to a patient's nostrils through a conduit, such as a cannula, that does not form an airtight seal with the patient's nostrils. A system in accordance with claimed subject matter may include a feature in a cannula, or other component that is inserted into a patient's nostrils, that ensures that an airtight seal is not formed between the patient's nostrils and the cannula. The opening thus-formed ensures that any escaping gas does not harm the patient's skin as gas escaping at relatively high speed through a leak between an otherwise tight-fitting mask and the patient's skin may. The patient's mouth is also free of encumbrances, unlike conventional systems that employ a full face mask that covers a patient's mouth and nostrils in an air-tight seal in an attempt to force air into the patient's respiratory system. An open system in accordance with claimed subject matter, a system that delivers respiratory gas to a patient's nostrils while allowing gas to escape the nostrils, provides for greatly improved patient comfort and safety and a concomitant improvement in patient compliance. A system in accordance with the principles of claimed subject matter may supply respiratory gas at a plurality of rates: higher rates for inspiratory air flow and lower rates for expiratory air flow, for example.

For treatment of obstructive breathing disorders, a system may supply respiratory gas at a high flow rate, that is, a flow rate sufficient to open the patient's respiratory passage, particularly the soft palate. A high flow rate may vary from 12 to 80 liters per minute (LPM), for example. The appropriate flow rate for a given patient may be determined, for example, by a titration process which determines a minimal flow rate required to establish and maintain an open airway for the patient. The operating flow rate may be set at a slightly higher rate than the minimum rate required to maintain an open airway in order to provide some operating margin (e.g., a margin of 2 LPM), for example. A mask-free approach to delivery of a respiratory gas improves a patient's comfort, relative to that experienced with a continuous positive airway pressure (CPAP) system, by eliminating the binding, cumbersome, somewhat claustrophobic, mask required by CPAP treatment. Additionally, because no mask is used and, therefore, leakage around a mask is not an issue, the painful side effects that may be associated with the use of a mask, such as the painful drying and cracking of skin around a point of leakage, are avoided and patient compliance may thereby be improved. Further improvements in comfort and compliance may be achieved by controlling the temperature and relative humidity of the supplied respiratory gas. Improved patient compliance yields a more effective treatment for obstructive breathing disorders.

Respiratory gases of various compositions may be used for high flow rate respiratory therapy in accordance with the principles of claimed subject matter. Although the composition of air is typically approximately 21% Oxygen, 78% Nitrogen, and 1% Argon, by volume, the term “air” will be used herein to refer to any mixture of respiratory gases other than pure Oxygen, including gases having a composition of from 15% to 100% Oxygen by volume, with concomitant adjustments in the Nitrogen percentage, for example. According to the principles of claimed subject matter, the temperature and humidity of respiratory gas supplied to a patient may be heated and humidified to closely match the temperature and humidity of gases within a healthy respiratory tract. The temperature of such gases may range between, 30° C. and 40° C., or in a preferred embodiment, between 34° C. and 39 C, or, in a still more preferred embodiment, between 36.0° C. and 38.0° C., at the point of delivery to a patient (at the tip of a cannula, for example). The relative humidity of such gases, at the point of delivery to a patient, may range from 80% to 100%, or in a preferred embodiment, between 85% and 100%, or in a still more preferred embodiment, between 95% and 100%.

In illustrative embodiments, a respiratory gas may be heated and humidified within a compressor that supplies propulsion to the gas, or within an auxiliary heating and/or humidifying component, for example. A conduit, which connects the compressor to a cannula inserted in a patient's nostrils, may also include a heating device to maintain a desired temperature throughout the length of the conduit. One or more sensors may be included within a system in accordance with the principles of claimed subject matter to monitor and control the temperature, flow rate, and/or humidity of respiratory gas supplied to a patient. In an illustrative embodiment such sensors may be located within a cannula or conduit proximate a patient's nostrils, for example. In an illustrative embodiment, such sensors may be located within a conduit close enough to a patient's nostrils so that the temperature or humidity of the respiratory gas will not change appreciably by the time it reaches the patient's nostrils, yet distant enough so that the patient's own temperature or humidity of exhaled air, does not interfere with accurate measurements of the supplied respiratory gas.

Temperature-, humidity-, and flow rate-controlled respiratory gas may be provided in accordance with the principles of claimed subject matter through a system of flexible tubing, cannula, and inserts, for example. Such a respiratory gas-supply system may be of a length to permit a patient ease of movement during sleep and may include a flexible conduit of a length from one to two meters, a cannula of a length of from one-fifth to one an one half meters, and nasal inserts adapted for insertion in the cannula at one end and into a patient's nostrils at the other end. Components, such as nasal cannula, or portions thereof, may be configured for ready replacement in accordance with principles of claimed subject matter. In this way, components, or portions thereof, which might be more susceptible to wear or to hygienic challenge may be replaced at lower cost than would be associated with replacement of an entire conduit/cannula system, for example.

In an illustrative embodiment in accordance with the principles of claimed subject matter, a respiratory gas supply system in accordance with the principles of claimed subject matter may supply gas to a patient at a plurality of rates. For example, a system may supply a respiratory gas at a low rate (for example, 0 to 10 LPM) when the patient is exhaling and at a higher rate (for example, 12 to 80 LPM) when the patient is inhaling. A system may alter the rate of flow based on a predetermined cadence: 1.5 seconds at a low flow rate, followed by 2.5 seconds at a high flow rate, or 1.5 seconds at a high flow rate, followed by 2.5 seconds at a low flow rate, for example. Or a system may employ continuous high frequency oscillation, for example. A system may also alter the rate at which respiratory gas is supplied in response to sensing a patient's biological activity, such as diaphragm activity or chest wall movement, for example. By controlling flow of respiratory gas in this manner a system in accordance with the principles of claimed subject matter may reduce the noise levels associated with the supply of respiratory gas. Additionally, multi-level respiratory gas supply provided by a system in accordance with the principles of claimed subject matter is particularly well-suited for patients suffering from acute or chronic respiratory failure, congestive heart failure, or other impairment that requires the use of supplemental respiratory gas.

### BRIEF DESCRIPTION OF THE FIGURES

Non-limiting and non-exhaustive embodiments will be described with reference to the following Figures, wherein like reference numerals refer to like parts throughout the various Figures unless otherwise specified.

FIG. 1 is a block diagram of a respiratory gas supply system in accordance with the principles of claimed subject matter;

FIG. 2 is a flow chart depicting an illustrative process of supplying respiratory gas in accordance with the principles of claimed subject matter;

FIG. 3 is a schematic diagram of an illustrative embodiment of a respiratory gas supply system in accordance with the principles of claimed subject matter;

FIGS. 4A through 4C are plan views of illustrative embodiments of respiratory air cannulas in accordance with the principles of claimed subject matter;

FIG. 5 is a block diagram of a processor that may be employed in an illustrative embodiment of a control system for a respiratory gas supply system in accordance with the principles of claimed subject matter.

### DETAILED DESCRIPTION

Although claimed subject matter will be described in terms of certain embodiments, other embodiments, including embodiments that do not provide all of the benefits and features set forth herein, are also within the scope of this invention. Various structural, logical, process step, and electronic changes may be made without departing from the spirit or scope of the invention. Flow charts may include steps that may be deleted or otherwise modified and the sequence set forth within a particular flow chart may be modified while keeping within the scope of the invention. Accordingly, the scope of the invention is defined only by reference to the appended claims.

In an apparatus and method in accordance with the principles of claimed subject matter, warm, humidified, respiratory gas may be supplied to a patient through an open delivery system for treatment of respiratory conditions, including obstructive sleep apnea, hypopnea, congestive heart failure, or respiratory failure, for example. Rather than forcing a respiratory gas into a patient's respiratory system by sealing a patient's breathing orifices (e.g., nostrils, and, in some cases, mouth) and forcing a gas under pressure into the patient's nostrils, as a conventional closed system (e.g., CPAP system) would, a system in accordance with the principles of claimed subject matter may supply a respiratory gas to a patient's nostrils through a cannula having open tips configured for insertion in a patient's nostrils. The cannula does not require a mask, nor does it form a gas-tight seal with the patient's nostrils. As a result, a patient may experience greater comfort and ease of breathing and more readily comply with respiration therapy. Because respiratory gas is supplied from an open delivery system (that is, respiratory gas is allowed to escape from the patient's nostrils) a patient may exhale more easily than with conventional closed delivery systems that don't allow respiratory gas to escape through a patient's nostrils during exhalation. A patient's comfort may be further enhanced in this manner and may be more compliant with his respiration therapy as a result. Additionally, an open system in accordance with the principles of claimed subject matter avoids the creation of painful sores caused by gas escaping through localized gaps between a mask and the patient's skin, as may occur with conventional closed (i.e., purportedly gas-tight) respiratory gas delivery systems.

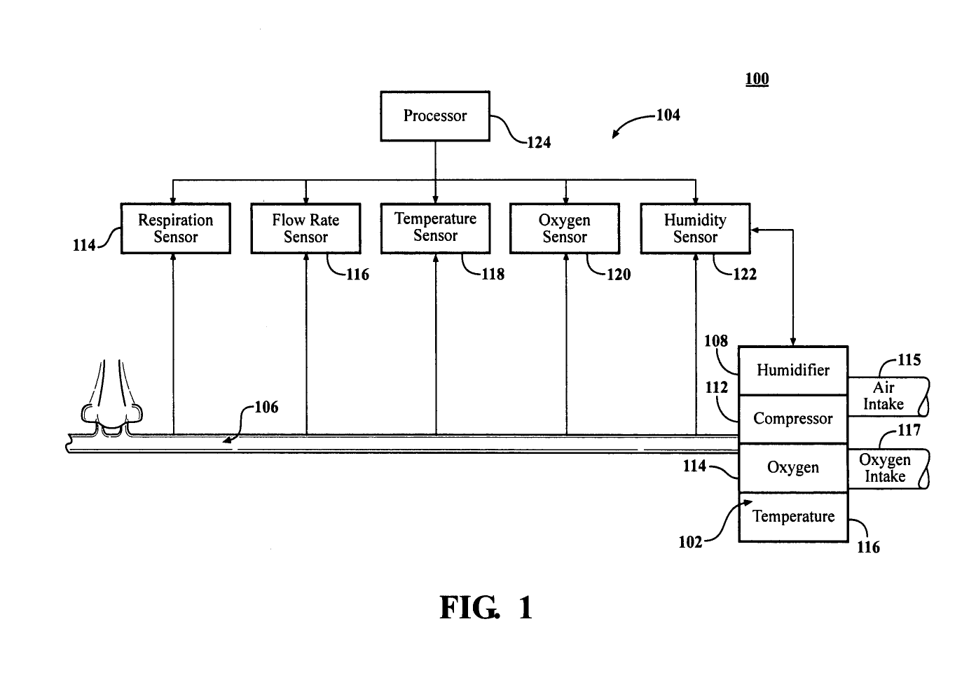

The block diagram of FIG. 1 includes components of an illustrative embodiment of an open delivery respiratory system 100 in accordance with the principles of claimed subject matter. The system 100 includes a respiratory gas conditioner 102, a controller 104, and a delivery component 106. Controller 104 may be implemented using a variety of technologies, including: analog circuitry, digital circuitry, hybrid components, logic arrays, or microprocessor technology, for example. The respiratory gas conditioner 102 may include a humidifier 108, a heater 110, a cooler 111, a compressor 112, or an Oxygen supply system 114. In illustrative embodiments a system 100 in accordance with claimed subject matter may include individual elements (e.g., heater 110, humidifier 108), gas conditioner 102 or combinations thereof. A controller 104 may include a respiration sensor 114, a flow sensor, a temperature sensor 118, an oxygen sensor 120, a humidity sensor 122, or a processor 124, for example. Valves, such as scissor valves, actuators, or servo-valves (not shown) may also be used to control gas flow. Sensors may be situated anywhere within the system and may be used to regulate respective respiratory gas characteristics. In an illustrative embodiment, a flow sensor may be positioned where respiratory gas enters the humidifier and/or where respiratory gas supply tubing meets the nasal cannula, for example.

In an illustrative embodiment, a temperature sensor 118 senses the temperature of respiratory gas and provides feedback to heater 110 and cooler 111 in order to regulate the temperature of respiratory gas to a range between 30° C. and 40° C., or in a preferred embodiment, between 34° C. and 39° C., or, in a still more preferred embodiment, between 36° C. and 38° C., at the point of delivery to a patient. A thermistor sensor may be used as temperature sensor 118, for example. As described in greater detail in the discussion related to the following Figures, a respiration sensor 114 may be employed by a respiratory system 100 to detect characteristics of a patient's breathing, such as the initiation of inhalation or exhalation, for example. Respiration sensors are known. A pneumatic belt breathing sensor is described in U.S. Pat. No. 4,602,643 issued to Henry G. Deitz, and piezo-electric belt sensors are known and available, for example, from iWorx/CB Sciences, One Washington Street, Suite 404, Dover N.H. 03820, and pyroelectric polymer (PEP) films have been proposed as transducers for respiratory rate monitors, for example. A zRIP respiratory inductance sensor belt may be obtained from Pro-Tech, online, at http://www.pro-tech.com/scripts/asp/prod_zrip.asp. Such sensors may detect movement of a patient's chest wall, for example.

Measurements obtained by respiration sensor 114 may be employed by the controller 104 to adjust the rate of flow of respiratory gas supplied by the system 100. For example, an indication from a respiration sensor 114 that a patient has begun to inhale may be used by the controller 104 to increase the flow of respiratory gas, or an indication from a respiration sensor 114 that a patient has begun to exhale may be used by the controller 104 to diminish, or even cut off entirely, the flow of respiratory gas. Respiratory gas flow rates sensors are described in, “Wireless Microsensor System for Monitoring a Breathing Activity”, IFMBE Proceedings, 4^{th} European Conference of the International Federation for Medical and Biological Engineering, by Jos Vander Sloten, Pascal Verdonck, Marc Nyssen, and Jens Hauesien, for example.

A flow sensor 116 may be used to determine the rate of flow of respiratory gas and, when compared with a target value in a feedback configuration, may be used to regulate the flow rate of the respiratory gas. In various illustrative embodiments, the flow rate may be a single, preset, value; may be multi-valued, with, for example, a low flow rate for a predetermined period (e.g., 1.5 seconds) followed by a high flow rate for a predetermined period (e.g., 2.5 seconds); or may be set at a high flow rate when a patient inhales and be set at a low flow rate when a patient exhales, for example. Continuous high frequency oscillation (CHFO), or high frequency oscillatory ventilation (HFOV), may also be employed by a system in accordance with the principles of claimed subject matter. CHFO is known and described, for example, in and article entitled, “High Frequency Oscillatory Ventilation” in “The Internet Journal of Emergency And Intensive Care Medicine 2003, Vol. 6, No. 2,” available at http://www.ispub.com/ostia/index.php?xmlFilePath=journals/ijeicm/vol6n2/hfov.xml. Combinations of such flow rate settings are contemplated within the scope of claimed subject matter. A high flow rate may vary from twelve to eighty liters per minute (LPM), for example. The appropriate flow rate for a given patient may be determined, for example, by a titration process which determines a minimal flow rate required to establish and maintain an open airway for the patient. The operating flow rate may be set at a slightly higher rate than the minimum rate required to maintain an open airway in order to provide some operating margin (e.g., a margin of two LPM), for example. A low flow rate may vary from zero to ten LPM. Respiratory gas may include air, received from air intake 115 or oxygen received through an oxygen intake 117 from an oxygen supply, such as a commercially available oxygen tank, for example. As described in greater detail below, various combinations of air and oxygen are contemplated within the scope of the claimed subject matter.

In illustrative embodiments a respiratory gas supply system 100 may determine gas supply flow rates by presetting a valve opening. That is, rather than measuring the flow rate of the respiratory gas and providing feedback to a controller 124, a respiratory gas supply system 100 in accordance with the principles of claimed subject matter may have flow rate and valve settings correlated, in a manufacturing or test setting, for example, so that flow rates may be determined by adjusting valves to predetermined settings. In such illustrative embodiments the flow rate may be a single rate, or may be adjustable to a plurality of rates, for example. As with a system that employs flow rate feedback, the flow rate may be set at a high level while a patient inhales and at a low level while the patient exhales, for example. Humidity sensor 122 may be employed in closed-loop feedback regulation of respiratory gas embodiments of a respiratory system 100 in accordance with the principles of claimed subject matter. Humidity sensors are known and described, for example, in U.S. Pat. No. 6,895,803, issued to Seakins et al, and entitled, Humidity Sensor. Oxygen sensor 120 may be used in illustrative closed-loop feedback embodiments of a respiratory gas supply system in accordance with the principles of claimed subject matter to regulate the percentage by volume of oxygen of supplied respiratory gas. Oxygen sensors are known and described, for example in U.S. Pat. No. 4,109,509 issued to Cramer et al and entitled, Oxygen Monitoring and Warning Device for Aircraft Breathing System.

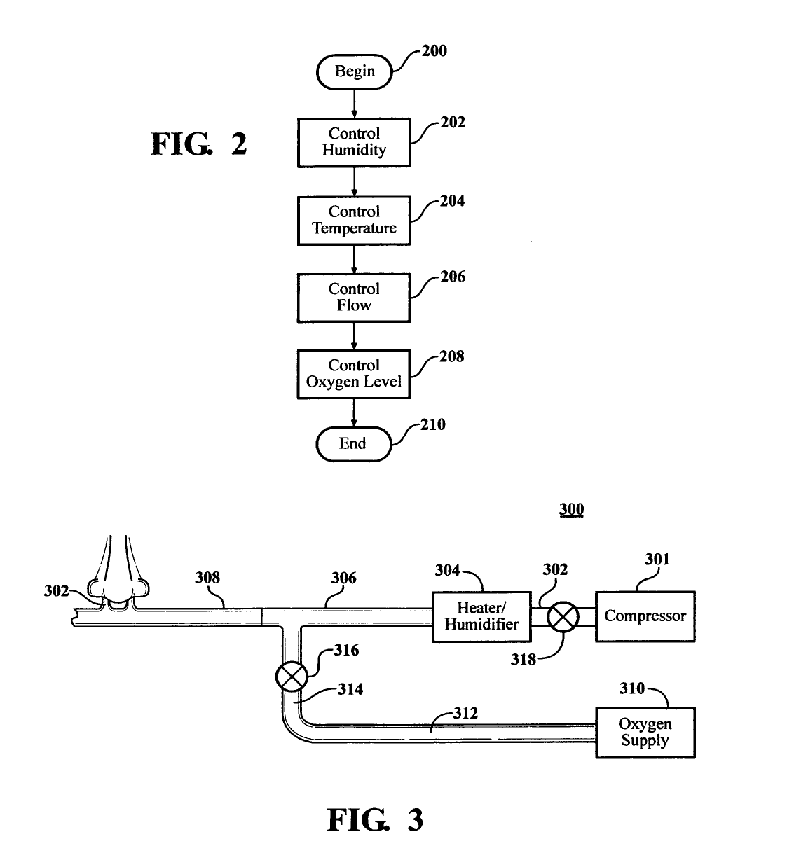

The flow chart of FIG. 2 outlines an illustrative process in accordance with the principles of claimed subject matter. Steps may be included that may be deleted or otherwise modified and the sequence set forth within the flow chart may be modified while keeping within the scope of claimed subject matter. Accordingly, the scope of the invention is defined only by reference to the appended claims.

The process begins in step 200 and proceeds from there to step 202 where the humidity of a respiratory gas to be supplied to a patient is controlled. In accordance with the principles of claimed subject matter, the control of respiratory gas may be carried out via a closed-loop or an open-loop process. A respiratory gas supply system in accordance with the principles of claimed subject matter may include a humidifier interface that permits the adjustment of the relative humidity of respiratory gases. In open-loop embodiments, adjustments at the interface are reflected in the humidifier mechanism to establish the target relative humidity. Adjustments of relative humidity settings may be calibrated during manufacturing or test, for example. In closed loop feedback embodiments, the relative humidity sensor, which may be located within a conduit that supplies a respiratory gases to the patient, provides an indication of the respiratory gas's relative humidity. That indication may be compared to a target relative humidity by a system controller, and the difference between target and measured relative humidities may be used to adjust the humidifier mechanism. The relative humidity of such gases, at the point of delivery to a patient, may range from 80% to 100%, or, in a preferred embodiment, between 85% and 100%, or, in a still more preferred embodiment, between 95% and 100%, for example.

In accordance with the principles of claimed subject matter, a membrane-free, heated-plate humidifier may be employed to humidify respiratory gases. A heated-plate humidifier may also be referred to as a nebulizer, for example. Membrane-based humidifiers may present special challenges, and, for that reason, they are not employed in a system and method in accordance with the principles of claimed subject matter.

To ensure accurate measurement of the relative humidity of respiratory gases supplied to a patient, a relative humidity sensor may be positioned proximate the distal end of a supply conduit, that is, at the end, nearest the patient. Systems for producing humidified respiratory gases and for sensing the humidity of respiratory gases are known and described, for example, in U.S. Pat. No. 4,238,425, entitled, Ultrasonic Humidifier, issued to Matsuoka et al, U.S. Pat. No. 7,111,624, entitled Apparatus For Delivering Humidified Gases, issued to Thudor, et al, U.S. Pat. No. 7,478,635, entitled Breathing Assistance Apparatus, issued to Wixey et al, U.S. Pat. No. 4,192,836, entitled, Respiratory Gas Humidifier, issued to Bartscher et al, and U.S. Pat. RE40,806, entitled Respiratory Humidification System, issued to Gradon, et al. Humidity from humidified respiratory gases may precipitate in a conduit carrying the gas to a patient. A gas conduit may be heated in order to counteract the tendency towards condensation or precipitation or a conduit may include a wick to carry away condensate; such conduits are known and described, for example, in U.S. Pat. No. 7,559,324, entitled Conduit With Heated Wick, issued to Smith et al.

From step 202 the illustrative process proceeds to step 204, where the temperature of the respiratory gas is controlled. In illustrative embodiments, the respiratory gas temperature may be controlled in an open loop or closed loop feedback system. In an open loop embodiment, the temperature may be preset, during manufacturing, for example, with adjustment afforded to a patient through a user interface, for example. In illustrative embodiments, the respiratory gas temperature may range between, 30° C. and 40° C., or in a preferred embodiment, between 34° C. and 39 C, or, in a still more preferred embodiment, between 36.0° C. and 38.0° C. In closed loop feedback embodiments, a temperature sensor may measure the temperature of respiratory gases and provide the temperature measurement to a controller, such as controller 114, which compares the measured temperature to a target temperature and adjusts a heater or cooler to regulate the temperature of the respiratory gas to within a desired temperature range. In order to ensure that the respiratory gas temperature is within a target range at the point of delivery, a temperature sensor may be located proximate to the distal end of the respiratory gas conduit.

As will be described in greater detail in the discussion related to the following figures, the gas conduit itself may include a heater and may be insulated to minimize changes in gas temperature as a respiratory gas flows through the conduit. Heated conduits for the delivery of respiratory gases are known and described, for example, in U.S. Pat. No. 7,588,029, entitled, Humidified Gas Delivery Apparatus, issued to Smith et al. A respiratory gas conduit that includes insulating material which serves to maintain the temperature of respiratory gases is disclosed in U.S. Pat. No. 7,637,288, entitled Respiratory Gas Hose System For Supplying A Respiratory Gas, issued to Kressierer/Huber et al.

From step 204 the process proceeds to step 206 where the respiratory gas supply system controls the flow of respiratory gas. Open loop or closed loop feedback control may be employed by a respiratory gas supply system in accordance with the principles of claimed subject matter. Open loop control may be achieved, for example, by correlating valve settings to flow levels during manufacturing or subsequent testing. Settings for a specific patient may then be determined through a titration process, for example. For patients suffering from obstructive breathing disorders, such a titration process may involve overnight observation of the patient, with adjustment of respiratory gas flow levels until an efficacious flow level is found. For patients suffering from congestive heart failure or respiratory failure, efficacious flow rates may be determined by supplying respiratory gas and monitoring the patient's blood oxygen level, for example. Closed-loop control may be implemented by including a flow sensor in a respiratory gas delivery system in accordance with claimed subject matter. In an illustrative embodiment, such a sensor may be included at the distal end of a respiratory gas supply conduit, proximate a patient's nostrils. Flow rates sensors are known and described, for example in U.S. Pat. RE40,806, entitled Respiratory Humidification System, issued to Gradon et al.

Appropriate flow rates may be determined by an auto-titration process by a system and method in accordance with the principles of claimed subject matter. Similar processes, used in positive airway pressure systems, are known and available in auto-titration positive airway pressure (APAP) systems, available, for example, from Resmed Corporation, 9001 Spectrum Center Boulevard, Sand Diego, Calif. 92123. However, because such systems are occluding systems, systems that rely upon a respiratory gas pressure buildup at the entrance to a patient's nostrils, adjustments to an auto-titration process must be made to adapt such a system to an open-deliver, non-occluding, system and method in accordance with the principles of claimed subject matter. That is, APAP systems detect and compensate for inadvertent leaks in their respiratory gas delivery systems. In contrast, an open-delivery, non-occluding system and method in accordance with the principles of claimed subject matter allows respiratory gas to escape a patient's nostrils, by design. For this reason, although a compressor such as that offered by Resmed may be employed, auto-titration is not contemplated within the scope of claimed subject matter.

In accordance with the principles of claimed subject matter, a respiratory gas supply system in accordance with the principles of claimed subject matter may provide respiratory gases at multiple flow rates. Although single flow rates may be effective in treating some conditions, multilevel flow rates may provide improved treatment in some instances. In the treatment of obstructive reading disorders a single flow rate may be set to maintain an open airway, or multiple levels may be employed to improve comfort, for example. That is, while a patient is inhaling, a relatively high flow rate may be established, and while the patient is exhaling, a relatively low flow rate may be employed. The relatively low flow rate provided during exhalation may improve a patient's comfort level by reducing the “back-pressure” experienced by a patient and thereby allowing a patient to exhale more easily. Operating at lower flow rates may also reduce the noise associated with the flowing respiratory gas, thereby further improving a patient's comfort level. For patients suffering from other respiratory conditions, such as respiratory failure or reduced cardiac function, multi-level flow rates may also improve a patient's comfort and respiration.

For treatment of obstructive breathing disorders, a system in accordance with the principles of claimed subject matter may supply respiratory gas at a high flow rate, that is, a flow rate sufficient to open the patient's respiratory passage, particularly the soft palate. A high flow rate may vary from 12 to 80 LPM, for example. The appropriate flow rate for a given patient may be determined, for example, by a titration process which determines a minimal flow rate required to establish and maintain an open airway for the patient. The operating flow rate may be set at a slightly higher rate than the minimum rate required to maintain an open airway in order to provide some operating margin (e.g., a margin of 2 LPM), for example. A mask-free, non-occluding, approach to delivery of a respiratory gas improves a patient's comfort, relative to that experienced with a continuous positive airway pressure (CPAP) system, by eliminating the binding, cumbersome, somewhat claustrophobic, mask required by CPAP treatment. Additionally, because no mask is used and, therefore, leakage around a mask is not an issue, the painful side effects that may be associated with the use of a mask, such as the painful drying and cracking of skin around a point of leakage, are avoided and patient compliance may thereby be improved. CPAP systems that eliminate masks but require occluding delivery (e.g., tight connection between cannula prongs and a patient's nostrils), although somewhat less cumbersome than mask-based CPAP systems, remain uncomfortable and may cause painful sores in areas of leakage around the cannula prongs.

A system in accordance with the principles of claimed subject matter may supply a respiratory gas at a low rate (for example, 0 to 10 LPM) when the patient is exhaling and at a higher rate (for example, 12 to 80 LPM) when the patient is inhaling. Scissor valves and actuators may be employed, for example, to effect flow resistance and to thereby regulate flow rates. Such application of scissor valves is known and described, for example, in “Flow Resistance of Expiratory Positive-Pressure Valve Systems,” Banner et al, Chest 1986; 90; 212-217. A system may alter the rate of flow based on a predetermined cadence (for example, 1.5 seconds at a low flow rate, followed by 2.5 seconds at a high flow rate). A system may also alter the rate at which respiratory gas is supplied in response to sensing a patient's biological activity, such as diaphragm activity, for example. Respiration monitors are known and described, for example, in U.S. Pat. No. 4,602,643, entitled, Pneumatic Breathing Belt Sensor With Minimum Space Maintaining Tapes, issued to Dietz, a pneumatic belt breathing sensor is described in U.S. Pat. No. 4,602,643 issued to Henry G. Deitz, and piezo-electric belt sensors are known and available, for example, from iWorx/CB Sciences, One Washington Street, Suite 404, Dover N.H. 03820, and pyroelectric polymer (PEP) films have been proposed as transducers for respiratory rate monitors, for example. By controlling flow of respiratory gas in this manner a system in accordance with the principles of claimed subject matter may reduce the noise levels associated with the supply of respiratory gas. Additionally, multi-level respiratory gas supply provided by a system in accordance with the principles of claimed subject matter is particularly well-suited for patients suffering from respiratory failure, congestive heart failure, or other impairment that requires the use of supplemental respiratory gas.

In accordance with the principles of claimed subject matter, a flow rate for treating obstructive respiratory disease may be established through a titration procedure at a level that ensures that a patient's breaths are not flow limited. Such a level may be manifested by a rounded inspiratory flow contour, an increase in peak inspiratory flow, a decline in supraglottic pressure swings, and the absence of snoring, for example. This flow rate may be set as a higher flow rate in an multi-level flow embodiment or, simply, as the single flow rate in a single flow rate embodiment. In multi-level flow rate embodiments, the lower flow rate, supplied while a patient exhales, may be reduced during a titration process until the patient's breaths become flow limited, then increased to a somewhat higher level to ensure that the patient's airway remains unblocked. Flow characteristics, such as a rounded inspiratory flow contour, an increase in peak inspiratory flow, and a decline in supraglottic pressure swings may be employed as indicators of therapeutic respiratory gas flow rates, for example. For some patients, the lower flow rate may be reduced to zero without their upper airway collapsing and, for these patients, the compressor supplying respiratory gas may be cycled off during their expiratory periods. For patients with obstructive breathing disorders, a system and method in accordance with claimed subject matter increases pharyngeal pressure and reduces ventilatory drive.

Not wishing to be bound by theory, it is believed that increased end-expiratory pharyngeal pressure provided by a respiratory gas delivery system and method in accordance with claimed subject matter increases upper airway patency, increases lung volume, improves oxygen stores and, because respiratory gas is delivered directly into a patient's nostrils, reduces dead space ventilation, all of which contribute to improved ventilation for patients suffering from obstructive respiratory conditions. For patients suffering from other conditions, such as respiratory or cardiac failure, it may be more important to reduce the flow of respiratory gases to a minimal level (e.g., down to 0 LPM) during the expiratory phase of their breathing cycle in order to further assist their ventilation.

From step 206 the process proceeds to step 208 where the oxygen level of respiratory gas is controlled. In accordance with the principles of claimed subject matter respiratory gases of various compositions may be used for respiratory therapy in accordance with the principles of claimed subject matter. Supplementary oxygen may be introduced into a flow of respiratory gas in accordance with the principles of claimed subject matter in order to provide respiratory gas having a percentage of oxygen that is greater than 21% by volume. As will be described in greater detail in the discussion related to the following Figures, a oxygen may be introduced through a “T” fitting into a respiratory gas conduit. The respiratory gas's percentage of oxygen may be controlled in open loop or closed loop embodiments in accordance with the principles of claimed subject matter. In closed loop feedback embodiments an oxygen level sensor may be positioned near the distal end of a respiratory gas supply conduit, for example. Oxygen-level sensors are known and described, for example, in U.S. Pat. RE40,806, entitled Respiratory Humidification System, issued to Gradon, et al. The composition of air is typically approximately 21% Oxygen, 78% Nitrogen, and 1% Argon, by volume, and respiratory gas supplied to a patient by a system and method in accordance with claimed subject matter may vary from in Oxygen content from ambient air to 100% oxygen, with higher levels of oxygen typically reserved for patients suffering from respiratory failure or cardiac failure, for example. From step 208 the process proceeds to end in step 210.

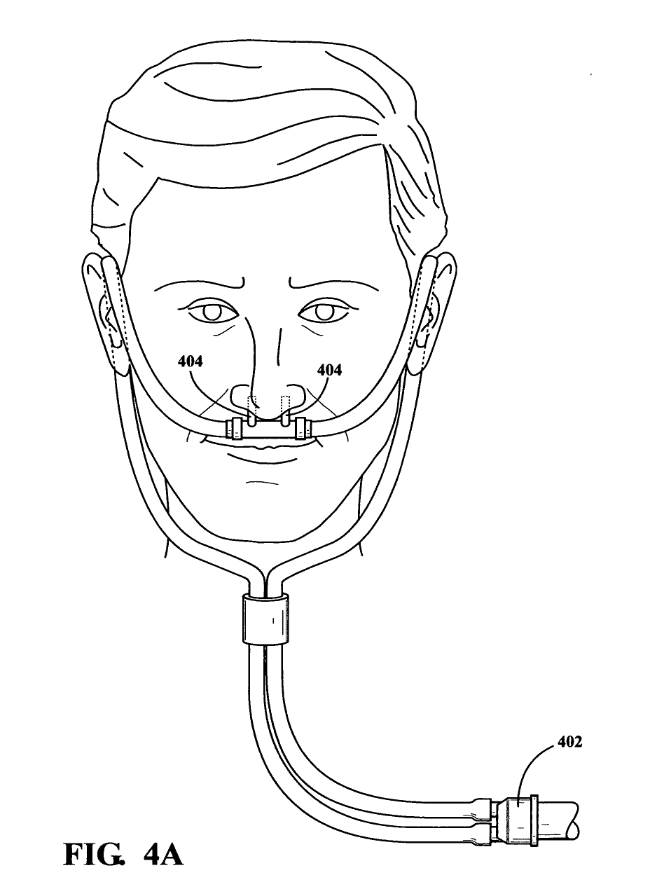

An illustrative embodiment of a respiratory gas supply system 300 in accordance with claimed subject matter is depicted in the mechanical system block diagram of FIG. 3. Compressor 300 provides pressurized respiratory gas through a conduit 302 to a heater/humidifier 304, where the respiratory gas is heated and humidified, as described in the discussion related to FIGS. 1 and 2. A flexible tube 306 (also referred to herein as a conduit) carries humidified, heated respiratory gas to open cannula 308. Oxygen may be supplied from an oxygen source 310 through a second conduit 312 coupled to conduit 306 via a Tee connection 314, for example. A variable valve 316, such as a scissor valve, may be operated to control the flow of oxygen into the conduit 306. Valve 318 may be operated to control the flow of respiratory gas from compressor 302 or humidifier 304. Valves 316 and 318 may be operated as previously described to control the flow rate of respiratory gas to the patient and to control the oxygen level (e.g., by volume) of respiratory gas supplied to a patient. Gas supply tube 306 may be of a length sufficient to allow a patient to move during sleep, for example. In illustrative embodiments supply tube 306 may be between one and two meters length, for example. Flexible tube 306 may be heated and/or insulated, as previously described, to prevent condensation of water within the tube. At least one prong 320 routes respiratory gas from cannula 308 to a patient's nostril(s). As previously described the prong 320 is designed to be non-occluding; in addition to supplying high flow-rate respiratory gas to a patient, it allows respiratory gas to readily escape a patient's nostril. An open-delivery approach such as this provides a patient with greater comfort than may be experienced while using an occluding system. Although respiratory gas is allowed to escape, in order to provide patient comfort, high respiratory gas flow rates in accordance with claimed subject matter ensures that the airway of an obstructive apnea patient will be opened.

Although employed in occluding systems, compressors 301, heaters and non-membrane humidifiers 304 are known. A compressor/heater/humidifier system having model number PM15 is available from Precision Medical, Inc. 300 Held Drive, Norhthampton', Pa. 18067. Autoset model 36005 is available from Resmed Corporation, 9001 Spectrum Center Boulevard, Sand Diego, Calif. 92123. Model DS750P is available from Phillips Respironics, Inc, 1010 Murry Ridge Lane, Murrysville, Pa. 15668-8525. A Fisher Paykel MR850 non-membrane humidifier may be used with a separate compressor or heater and may be obtained from Fisher & Paykel Healthcare, Inc., 15365 Barranca Parkway, Irvine, Calif. 92618. A system referred to as “Bipap Vision” system, available from Phillips Respironics (http://bipapvision.respironics.com/) may also be used in an open delivery system in accordance with the principles of claimed subject matter.

As previously indicated, conventional membrane-free, or heated-plate, humidifying systems, such as the Bipap Vision system, occlude a patient's nares, then pressurize a respiratory gas to force the gas into the patient's airways. In order to take advantage of the compressor, heater, and humidifier of a system such as the Bibpap Vision system, while employing a non-occluding, open-nares, approach in accordance with the principles of claimed subject matter a conversion from pressure-control to flow control may be implemented in a number of ways. Flow rates for a given open-cannula embodiment may be correlated with different pressure settings, using an existing pressure-based interface, for example. One or more flow sensors may be placed in the respiratory gas supply line, between a compressor and humidifier, and/or proximate the patient-end of a respiratory gas supply tube, with readout for manual adjustment, or with closed-loop control for automated adjustment of flow rates, as previously described. Although existing compressors, humidifiers, and heaters may be employed in a system in accordance with the principles of claimed subject matter, systems capable of maintaining humidification at higher flow rates are contemplated within the scope of claimed subject matter.

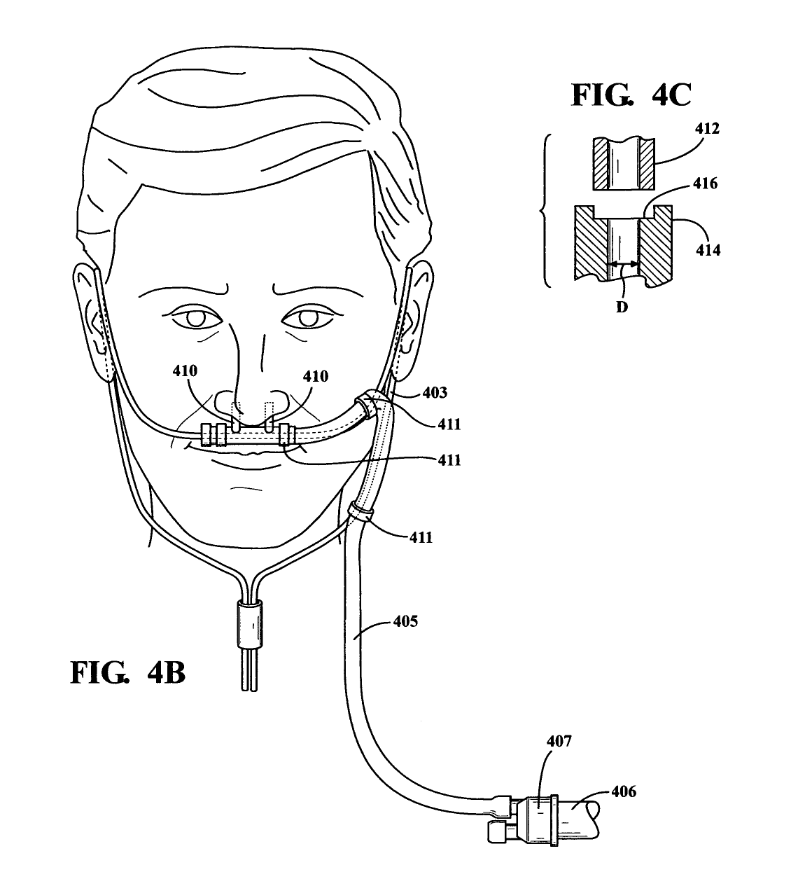

In an illustrative embodiment, a cannula 400, of FIG. 4A includes a mating connector for connection to a conduit such as conduit 306 of FIG. 3. The total length of the cannula may vary from patient to patient, but will typically be long enough to allow the cannula to be draped over patient's ears for support. In illustrative embodiments, the length of the cannula 400 varies from one-one third to two meters. The inside diameter of the cannula 400 may be such that the cross-section is half the cross-section of the conduit to which the cannula is attached. Cannula 400 may be made of flexible tubing material and, in an illustrative embodiment, has an inside diameter, D, of between five and twelve millimeters. Tubing that supplies respiratory gas to the cannula 400 may have an inside diameter of between twelve and thirty millimeters in an illustrative embodiment. In this manner, flow is not restricted in the transition from tubing 306 to cannula 400. In illustrative embodiments, cannula 400 and tubing 306 may be formed of one piece or may include a plurality of connected segments. Cannula 400 and tubing 306 may include heating elements and/or insulation, as previously described, to prevent condensation, also referred to herein as “rainout.” Because cannula 400 may have more intimate contact with a patient, it may be advantageous to replace cannula 400 more frequently than tubing 306. Multi-segment embodiments allow cannula 400 to be replaced, while retaining tubing 306. In this illustrative embodiment prongs for insertion in a patient's nose are situated approximately midway along the cannula 400. In illustrative embodiments the prongs range from 5 mm to 30 mm in length. Inside diameters of the nasal prongs 404 are chosen to allow for free flow of respiratory gas and their total cross-section may therefore be of a size to approximate the cross-section of the conduit 306 with which the cannula is connected in this illustrative embodiment. Prongs 404 may be flared at the tip in order to reduce the noise associated with flowing respiratory gas. In no case, however, are the outside diameters of both prongs great enough to occlude a patient's nostril.

Rather than splitting the flow of respiratory gas between two paths, as conventional cannula do, an open nasal cannula in accordance with the principles of claimed subject matter may employ a single path in order to maximize gas flow. In the illustrative embodiment of FIG. 4B an open nasal cannula includes a single conduit 406 having an attachment mechanism 407 at its proximal end configured for attachment to respiratory gas conduit 306. At its distal end, the cannula 405 includes prongs 410 for insertion into a patient's nostrils. In an illustrative embodiment, the inside diameter of the cannula may be between five and twelve millimeters in order to accommodate high respiratory gas flow rates, for example. A cannula with large inside diameter may permit delivery of respiratory gas at high flow rates while requiring less work of a compressor than would be required with a cannula of smaller cross section. Additionally, a cannula with larger cross section may reduce noise associated with high flow-rate delivery of respiratory gases. In an illustrative embodiment attachment means 411, such as clamps or straps, for example, may be integrated with, or attached to, a cannula 405 in accordance with the principles of claimed subject matter. Attachment means 411 may be used along with a support device 413, such as a lanyard, that may be used to hold the cannula 405 in position to facilitate delivery of respiratory gas to a patient's nostrils. In an illustrative embodiment, support device 413 may be composed of a length of three-millimeter rubber tubing, for example, and may be draped over a patient's ears to support cannula 405. An adjuster 423, which may be a friction-fit device, for example, may be used to adjust snugness of fit of the length of tubing as the tubing loops around or otherwise engages with a patient's head. In such an illustrative embodiment the cannula may be firmly held in place without the discomfiture associated with conventional masks or the tendency of conventional cannulas to move out of place. Additionally, in such an embodiment, one side of the mouth is left relatively open, with no cannula loop on the other side of a patient's face. Without the obstruction of a cannula, a patient may find it easier to perform regular tasks, such as eating, drinking, or talking on the telephone, for example.

In an illustrative embodiment, nasal prongs such as prongs 410 may include inserts 412, which may be replaced on a regular basis in order to maintain patient hygiene, for example. Inserts 412 may be held in cannula receptacles 414 by friction fitting, for example. In order to ensure smooth flow of respiratory gases and to reduce noise associated with the flow of respiratory gases, the inside diameters of inserts 412 may be matched to the inside diameters of shoulders 416 within cannula receptacles 414, as illustrated in FIG. 4C. Cannula tips may include structures 418, such as bumps, for example, that are configured to maintain an opening between cannula tips and a patient's nostrils, thereby allowing gases escape from a patient's nostrils. As previously described, allowing gas to escape may increase a patient's comfort, particularly during the expiratory phase of a patient's breathing cycle, and may prevent painful sores associated with gas escaping from respiratory masks.

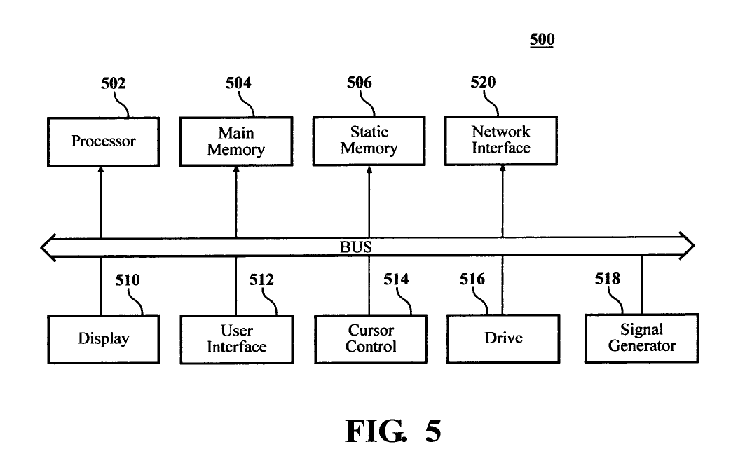

FIG. 5 is a schematic diagram of an illustrative embodiment of a processor 500 which may employed by a respiratory gas supply system in accordance with the principles of claimed subject matter. Processor 500 includes a processor 502 (e.g., a processor core, a microprocessor, a computing device, etc), a main memory 504 and a static memory 506, which communicate with each other via a bus 508. The processor 500 may further include a display unit 510 that may comprise a touch-screen, or a liquid crystal display (LCD), or a light emitting diode (LED) display, or a cathode ray conduit (CRT), for example. As shown, processor 500 also includes a human input/output (I/O) device 512 (e.g., a keyboard, an alphanumeric keypad, etc), a pointing device 514 (e.g., a mouse, a touch screen, etc), a drive unit 516 (e.g., a disk drive unit, a CD/DVD drive, a tangible computer readable removable media drive, an SSD storage device, etc), a signal generation device 518 (e.g., a speaker, an audio output, etc), and a network interface device 518 (e.g., an Ethernet interface, a wired network interface, a wireless network interface, a propagated signal interface, etc).

All or part of the processor 500, and the processes and methods as further described herein, may be implemented using or otherwise including hardware, firmware, software, or any combination thereof. By way of example but not limitation, processor 500 may include one or more processors, controllers, microprocessors, microcontrollers, application specific integrated circuits, digital signal processors, programmable logic devices, field programmable gate arrays, and the like, or any combination thereof. Processor 500 may include an operating system, one or more applications, and one or more drivers. It is to be understood that other embodiments may be used, for example, or changes or alterations, such as structural changes, may be made. All embodiments, changes or alterations, including those described herein, are not departures from scope with respect to intended claimed subject matter.

Reference throughout this specification to “one embodiment” or “an embodiment” means that a particular feature, structure, or characteristic described in connection with the embodiment is included in at least one embodiment of claimed subject matter. Thus, the appearances of the phrase “in one embodiment” or “an embodiment” in various places throughout this specification are not necessarily all referring to the same embodiment. Furthermore, the particular features, structures, or characteristics may be combined in one or more embodiments. While there has been illustrated and described what are presently considered to be example embodiments, it will be understood by those skilled in the art that various other modifications may be made, and equivalents may be substituted, without departing from claimed subject matter. Additionally, many modifications may be made to adapt a particular situation to the teachings of claimed subject matter without departing from the central concept described herein. Therefore, it is intended that claimed subject matter not be limited to the particular embodiments disclosed, but that such claimed subject matter may also include all embodiments falling within the scope of the appended claims, and equivalents thereof.

1. A method, comprising the steps of:
regulating the humidity of a respiratory gas using a heated plate humidifier; and
supplying the humidified respiratory gas to a patient through a high flow rate open conduit inserted into a patient's nostril.

2. The method of claim 1 further comprising the step of regulating the Oxygen level of the respiratory gas.

3. The method of claim 1 wherein the conduit includes a proximal end for attachment to a respiratory gas supply and the method further comprises the step of sensing the humidity of the respiratory gas near the distal end of the conduit.

4. The method of claim 1 further comprising the step of sensing the temperature of the respiratory gas near the distal end of the conduit.

5. The method of claim 1 further comprising the step of sensing the flow rate of the respiratory gas near the distal end of the conduit.

6. The method of claim 1 further comprising the step of sensing the Oxygen level within the respiratory gas near the distal end of the conduit.

7. The method of claim 1, further comprising the step of regulating the flow rate of the respiratory gas.

8. The method of claim 7, further comprising the step of sensing a biological function indicative of the patient's breathing; and
regulating the flow rate of respiratory gas responsive to the sensed biological function.

9. The method of claim 8 wherein the step of sensing a biological function includes sensing movement of a patient's chest wall.

10. A method, comprising the steps of:
regulating the humidity of a respiratory gas using a heated plate humidifier;
regulating the temperature of a respiratory gas; and
supplying the humidified respiratory gas to a patient through a high flow rate conduit inserted into a patient's nostril, wherein the inserted conduit is configured to allow respiratory gas to escape the patient's nostril.

11. The method of claim 10 further comprising the step of regulating the Oxygen level of the respiratory gas.

12. The method of claim 10 further comprising the step of sensing the humidity of the respiratory gas proximate the distal end of the conduit.

13. The method of claim 10 further comprising the step of sensing the temperature of the respiratory gas proximate the distal end of the conduit.

14. The method of claim 10 further comprising the step of sensing the flow rate of the respiratory gas proximate the distal end of the conduit.

15. The method of claim 10 further comprising the step of sensing the Oxygen level within the respiratory gas proximate the distal end of the conduit.

16. The method of claim 10 further comprising the step of sensing a biological function indicative of the patient's breathing.

17. The method of claim 16 wherein the step of sensing a biological function includes sensing movement of a patient's chest wall.

18. A method, comprising the steps of:
regulating the humidity of a respiratory gas using a heated plate humidifier;
regulating the temperature of a respiratory gas; and
supplying the humidified respiratory gas to a patient at a regulated flow rate through a high flow rate conduit inserted into a patient's nostril, wherein the inserted conduit is configured to allow respiratory gas to escape the patient's nostril.

19. The method of claim 18 wherein the flow rate of the respiratory gas is regulated at a plurality of discrete levels, including a high flow rate and a low flow rate.

20. The method of claim 19 wherein the high flow rate ranges between twelve and eighty liters per minute (LPM).

21. The method of claim 19 wherein the low flow rate ranges between zero and ten LPM.

22. The method of claim 19 wherein the flow rate is alternated between high and low flow rates according to a timed sequence.

23. The method of claim 22 wherein the timed sequence is adjusted for compatibility with a patient's breathing cycle.

24. The method of claim 23, further comprising:
determining the duration of a patient's inhalation and exhalation period over the course of a plurality of breathing cycles and regulating the high and low flow rates to substantially coincide, respectively, with the patient's inhalation and exhalation periods.

25. The method of claim 19, further comprising:
sensing a biological function of a patient indicative of a patient's breathing pattern; and
correlating the flow of respiratory gas with the patient's breathing pattern.

26. The method of claim 25 wherein the step of sensing a biological function includes sensing movement of a patient's chest wall.

27. The method of claim 25, further comprising:
increasing the flow of respiratory gas when a patient's initiation of an inhalation is sensed.

28. The method of claim 25, further comprising:
decreasing the flow of respiratory gas when a patient's initiation of an exhalation is sensed.

29. The method of claim 18 wherein the temperature is regulated to temperature between 30° C. and 40° C.

30. The method of claim 29 wherein the temperature is regulated to a temperature between 34° C. and 39° C.

31. The method of claim 30 wherein the temperature is regulated to a temperature between 36° C. and 38° C.

32. The method of claim 18 wherein the humidity is regulated to value between 80% and 100% relative humidity.

33. The method of claim 32 wherein the humidity is regulated to a value between 85% and 100% relative humidity.

34. The method of claim 33 wherein the humidity is regulated to a value between 95% and 100% relative humidity.

35. The method of claim 16 wherein the percentage of Oxygen is regulated to a value between 21% and 100% by volume.

36. The method of claim 35 wherein the percentage of Oxygen is regulated to a value between 21% and 80% by volume.

37. The method of claim 36 wherein the percentage of Oxygen is regulated to a value between 21% and 60% by volume.

38. An apparatus, comprising:
a heated-plate respiratory gas humidifier;
a high flow rate respiratory gas compressor; and
a high flow respiratory gas conduit, the compressor, and humidifier combined to supply humidified respiratory gas through the conduit at a high rate of flow to a patient, wherein the conduit includes prongs for insertion within a patient's nostrils for open delivery of respiratory gas.

39. The apparatus of claim 38 further comprising:
an oxygen source connected to mix oxygen with ambient air and to thereby delivery super-oxygenated respiratory gas to a patient.

40. The apparatus of claim 38 further comprising:
a temperature controller connected to maintain the temperature of the respiratory gas to within a range between 34° C. and 39° C.

41. The apparatus of claim 38 further comprising:
a flow rate controller connected to supply respiratory gas

42. The apparatus of claim 41 wherein the flow rate controller is configured to supply respiratory gas at a plurality of rates.

43. The apparatus of claim 42 further comprising a sensor connected to sense a patient's biological function and to regulate the flow of respiratory gas responsive to the sensed biological function.

44. The apparatus of claim 43 wherein the sensor is a chest wall sensor configured to sense movement of a patient's chest wall and to supply a relatively high flow rate of respiratory gas to the patient while the patient inhales and a relatively low flow rate or respiratory gas to the patient while the patient exhales.

45. The apparatus of claim 42 wherein the flow rate controller supplies respiratory gas at a high flow rate ranging between twelve and eighty liters per minute (LPM).

46. The apparatus of claim 42 wherein the flow rate controller supplies respiratory gas at a low flow rate ranging between zero and ten LPM.

47. The apparatus of claim 42 wherein the flow rate controller supplies respiratory gas at different flow rates according to a timed sequence.

48. The apparatus of claim 38 wherein the humidifier is configured to regulate the respiratory gas humidity to within a range between 80% and 100% relative humidity.

49. The apparatus of claim 48 wherein the humidifier is configured to regulate the respiratory gas humidity to within a range between 95% and 100% relative humidity.

50. The apparatus of claim 39 wherein the oxygen source is connected to supply respiratory gas with a percentage of oxygen falling within a range of 21% and 100% by volume.

51. An apparatus, comprising:
at least twelve millimeters in diameter; and
a prong of at least four millimeters inside diameter and less than fifty millimeters in length for insertion into a nostril of a patient, to thereby provide open delivery of respiratory gas from the flexible tube to a patient.

52. The apparatus of claim 51 wherein the inside diameter of the flexible tube is between twelve and thirty millimeters.

53. The apparatus of claim 52, wherein the inside diameter of the flexible tube is substantially constant throughout the length of the tube.

54. The apparatus of claim 53, further comprising a lumen formed at the distal end of the flexible tube, with the prong located along the lumen.

55. The apparatus of claim 53, wherein the lumen forms a loop with the prong positioned approximately midway along the length of the lumen, a cross section of the lumen being greater than half the cross-section of the tube.

56. The apparatus of claim 53, wherein the lumen terminates at a distal end and the prong is positioned substantially near the distal end.

57. The apparatus of claim 53, further comprising:
a heated-plate respiratory gas humidifier;
a high flow rate respiratory gas compressor; and the compressor, and humidifier combined to supply humidified respiratory gas through the flexible tube at a high rate of flow to a patient.

58. The apparatus of claim 57 further comprising:
an oxygen source connected to mix oxygen with ambient air and to thereby delivery super-oxygenated respiratory gas to a patient. Not sure what you mean here.

59. The apparatus of claim 57 further comprising:
a temperature controller connected to maintain the temperature of the respiratory gas to within a range between 34° C. and 39° C.

60. The apparatus of claim 57 further comprising:
a flow rate controller connected to supply respiratory gas

61. The apparatus of claim 57 wherein the flow rate controller is configured to supply respiratory gas at a plurality of rates.

62. The apparatus of claim 61 further comprising a sensor connected to sense a patient's biological function and to regulate the flow of respiratory gas responsive to the sensed biological function.

63. The apparatus of claim 62 wherein the sensor is a chest wall sensor configured to sense movement of a patient's chest wall and to supply a relatively high flow rate of respiratory gas to the patient while the patient inhales and a relatively low flow rate or respiratory gas to the patient while the patient exhales.

64. The apparatus of claim 61 wherein the flow rate controller supplies respiratory gas at a high flow rate ranging between twelve and eighty liters per minute (LPM).

65. The apparatus of claim 61 wherein the flow rate controller supplies respiratory gas at a low flow rate ranging between zero and ten LPM.

66. The apparatus of claim 61 wherein the flow rate controller supplies respiratory gas at different flow rates according to a timed sequence.

67. The apparatus of claim 57 wherein the humidifier is configured to regulate the respiratory gas humidity to within a range between 80% and 100% relative humidity.

68. The apparatus of claim 67 wherein the humidifier is configured to regulate the respiratory gas humidity to within a range between 95% and 100% relative humidity.

69. The apparatus of claim 61 wherein the oxygen source is connected to supply respiratory gas with a percentage of oxygen falling within a range of 21% and 100% by volume.
